(19) United States
(12) Patent Application Publication
(10) Pub. No.: US 2003/0229260 A1
(43) Pub. Date: Dec. 11, 2003

(54) Cardiac support device with tension indicator

(75) Inventors: Michael J. Girard, Lino Lakes, MN (US); Kay Ann Nauertz, South St. Paul, MN (US); Jose F. Nunez, St. Anthony, MN (US); James Cox, Hamel, MN (US); Holly Hicks, West St. Paul, MN (US)

Correspondence Address: ACORN CARDIOVASCULAR, INC., P.O. BOX 2903, MINNEAPOLIS, MN, 55402, US

(73) Assignee: Acorn Cardiovascular, Inc.

(21) Appl. No.: 10/163,597

(22) Filed: Jun. 5, 2002

(51) Int. Cl. A61N 1/362

(52) U.S. Cl. 600/16

(57) ABSTRACT

An apparatus for treating congestive heart disease and related cardiac complications such as valvular disorders that includes a support device placed on the heart. Support devices such as jackets of flexible material of knit construction defining a volume between an open upper end and a lower end are included in the invention. Such support devices can be dimensioned and sized based in part on the desired function of the device. The support device includes at least one indicator marker along the longitudinal axis of the device for indicating and/or monitoring the level of tension of the device or for selecting a predetermined level of tension of the support device.

### BACKGROUND OF THE INVENTION

1. Field of the Invention

The present invention pertains to a method and apparatus for treating congestive heart disease and related valvular dysfunction. More particularly, the present invention is directed to a cardiac support device having an indicator to monitor tensioning of the device on a heart and a method of using the device.

2. Description of the Prior Art

Congestive heart disease is a progressive and debilitating illness, characterized by a progressive enlargement of the heart. As the heart enlarges, it must perform an increasing amount of work in order to pump blood during each heartbeat. In time, the heart becomes so enlarged that it cannot adequately supply blood to the body. An afflicted patient is fatigued, unable to perform tasks requiring even a minimum level of exertion and experiences pain and discomfort. Further, as the heart enlarges, the internal heart valves may not adequately close. This impairs the function of the valves and further reduces the heart's ability to supply blood to the body.

Causes of congestive heart disease are not fully known. In some instances, congestive heart disease may result from certain viral infections. In such cases, the heart may enlarge to such an extent that even after the viral infection has passed the disease continues its progressively debilitating course. Congestive heart disease and treatment methodologies are described, for example in U.S. Pat. No. 6,123,662, issued Sep. 26, 2000 entitled CARDIAC DISEASE TREATMENT AND DEVICE, the disclosure of which is incorporated herein by reference.

Congestive heart failure has an enormous societal impact. In the United States alone, about five million people suffer from the disease. Alarmingly, congestive heart failure is one of the most rapidly accelerating diseases (about 400,000 new patients in the United States each year). Economic costs of the disease have been estimated at $38 billion annually.

One type of treatment can be found in commonly assigned U.S. Pat. No. 5,702,343 to Alferness dated Dec. 30, 1997. It teaches a jacket that constrains cardiac expansion during diastole. Also, PCT International Publication No. WO 98/29401 published Jul. 9, 1998 teaches a cardiac constraint in the form of surfaces on opposite sides of the heart with the surfaces joined together by a cable through the heart or by an external constraint. U.S. Pat. No. 5,800,528 dated Sep. 1, 1998 teaches a passive girdle to surround a heart. German utility model DE 295 17 393 describes a non-expansible heart pouch. PCT International Publication No. WO 98/58598 published Dec. 30, 1998 describes a cardiac pouch with an elastic limit.

A cardiac support device can be placed on an enlarged heart and fitted snug during diastole. For example, a knit jacket device can be loosely slipped on the heart. After such placement, the material of the jacket can be gathered to adjust the device to a desired tension. The gathered material can be sutured or otherwise fixed to maintain the tensioning. The heart may be pre-shrunk prior to placement of the device or the device may be fitted on the heart without pre-shrinking the heart. The device is adjusted for a snug fit on the heart during diastole.

Commonly assigned U.S. Pat. No. 6,174,279 teaches a cardiac constraint, which contains indicators that indicate jacket tensioning. The device disclosed therein can differentiate between the relaxed state and the tensioned state of the cardiac support device.

The present invention includes an improved device and method that allows the cardiac support device to be tensioned to different levels.

### SUMMARY OF THE INVENTION

According to one embodiment of the present invention, a method and apparatus are disclosed for treating congestive heart disease and related cardiac complications such as valvular disorders. According to the invention, a cardiac support device, such as a jacket of flexible material that defines a volume between an open upper end and a lower end is placed on the heart. Such a support device can be dimensioned and sized based in part on the desired function of the support device. The support device includes an indicator having markers along its longitudinal axis for monitoring the tensioning of the support device. In one embodiment, the support device can also include markers perpendicular to the longitudinal axis. Another embodiment has at least two longitudinal markers, at least one indicating a state of non-tension and at least one indicating a predetermined amount of tensioning.

### BRIEF DESCRIPTION OF THE DRAWINGS

FIG. 1 is a schematic cross-sectional view of a normal, healthy human heart shown during systole;

FIG. 1A is the view of FIG. 1 showing the heart during diastole;

FIG. 2 is a schematic cross-sectional view of a diseased human heart shown during systole;

FIG. 2A is the view of FIG. 2 showing the heart during diastole;

FIG. 3 is a perspective view of one embodiment of a cardiac support device;

FIG. 3A is a side elevation view of a diseased heart in diastole with the cardiac support device of FIG. 3 in place;

FIG. 4 is a perspective view of an alternative cardiac support device;

FIG. 4A is a side elevation view of a diseased heart in diastole with the device of FIG. 4 in place;

FIG. 5 is a cross-sectional view of the device of FIG. 3 overlying a myocardium and with the material of the device gathered for a snug fit;

FIG. 6 is a side elevation view showing a cardiac support device in place on a heart prior to tensioning with visually perceptible tension indicator grids of the present invention in a relaxed state;

FIG. 7 is a side elevation view of the device of FIG. 6 following the tensioning of the device with the visually perceptible tension indicator grids of the present invention in a tensioned state;

FIG. 8 is a side elevation view showing a cardiac support device in place on a heart prior to tensioning with visually perceptible baseline and fit line markers of the present invention in a relaxed state;

FIG. 9 is a side elevation view of the device of FIG. 8, wherein the cardiac support device is an open structure;

FIG. 10 is a side elevation view of the device of FIGS. 8 or 9 after tensioning (and/or cutting) of the material of the cardiac support device to the left of the baseline indicator line of the present invention, to conform the cardiac support device to the surface of the heart without any tension on the cardiac support device;

FIG. 11 is a side elevation view of the device of FIG. 10 after tensioning (and/or cutting) of the material of the cardiac support device between the baseline and a fit line indicator of the present invention, to tension the cardiac support device; and

FIG. 12 is a side elevation view showing a cardiac support device in place on a heart prior to tensioning with visually perceptible baseline and two fit line markers of the present invention in a relaxed state.

### DESCRIPTION OF THE PREFERRED EMBODIMENT

A. Congestive Heart Disease

To facilitate a better understanding of the present invention, description will first be made of one type of cardiac support device, a cardiac constraint device such as is described in U.S. Pat. No. 6,085,754 issued Jul. 11, 2000, the disclosure of which is incorporated herein by reference. In the drawings, similar elements are labeled similarly throughout.

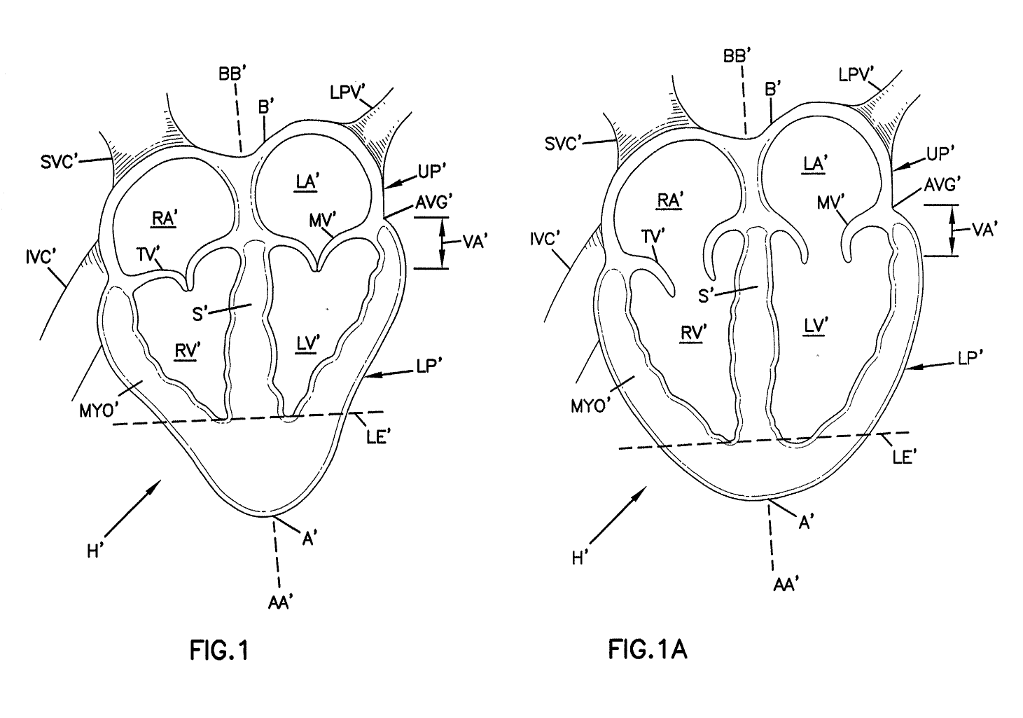

With initial reference to FIGS. 1 and 1A, a normal, healthy human heart H′ is schematically shown in cross-section and will now be described in order to facilitate an understanding of the present invention. In FIG. 1, the heart H′ is shown during systole (i.e., high left ventricular pressure). In FIG. 1A, the heart H′ is shown during diastole (i.e., low left ventricular pressure).

The heart H′ is a muscle having an outer wall or myocardium MYO′ and an internal wall or septum S′. The myocardium MYO′ and septum S′ define four internal heart chambers including a right atrium RA′, a left atrium LA′, a right ventricle RV′ and a left ventricle LV′. The heart H′ has a length measured along a longitudinal axis BB′-AA′ from an upper end or base B′ to a lower end or apex A′.

The right and left atria RA′, LA′ reside in an upper portion UP′ of the heart H′ adjacent the base B′. The right and left ventricles RV′, LV′ reside in a lower portion LP′ of the heart H′ adjacent the apex A′. The ventricles RV′, LV′ terminate at ventricular lower extremities LE′ adjacent the apex A′ and spaced there from by the thickness of the myocardium MYO′.

Due to the compound curves of the upper and lower portions UP′, LP′, the upper and lower portions UP′, LP′ meet at a circumferential groove commonly referred to as the A-V (atrio-ventricular) groove AVG′. Extending away from the upper portion UP′ are a plurality of major blood vessels communicating with the chambers RA′, RV′, LA′, LV′. For ease of illustration, only the superior vena cava SVC′, inferior vena cava IVC′ and a left pulmonary vein LPV′ are shown as being representative.

The heart H′ contains valves to regulate blood flow between the chambers RA′, RV′, LA′, LV′ and between the chambers and the major vessels (e.g., the superior vena cava SVC′, inferior vena cava IVC′ and a left pulmonary vein LPV′). For ease of illustration, not all of such valves are shown. Instead, only the tricuspid valve TV′ between the right atrium RA′ and right ventricle RV′ and the mitral valve MV′ between the left atrium LA′ and left ventricle LV′ are shown as being representative.

The valves are secured, in part, to the myocardium MYO′ in a region of the lower portion LP′ adjacent the A-V groove AVG′ and referred to as the valvular annulus VA′. The valves TV′ and MV′ open and close through the beating cycle of the heart H′.

FIGS. 1 and 1A show a normal, healthy heart H′ during systole and diastole, respectively. During systole (FIG. 1), the myocardium MYO′ is contracting and the heart H′ assumes a shape including a generally conical lower portion LP′. During diastole (FIG. 1A), the heart H′ is expanding and the conical shape of the lower portion LP′ bulges radially outwardly (relative to axis AA′-BB′).

The motion of the heart H′ and the variation in the shape of the heart H′ during contraction and expansion is complex. The amount of motion varies considerably throughout the heart H′. The motion includes a component that is parallel to the axis AA′-BB′ (conveniently referred to as longitudinal expansion or contraction). The motion also includes a component perpendicular to the axis AA′-BB′ (conveniently referred to as circumferential expansion or contraction).

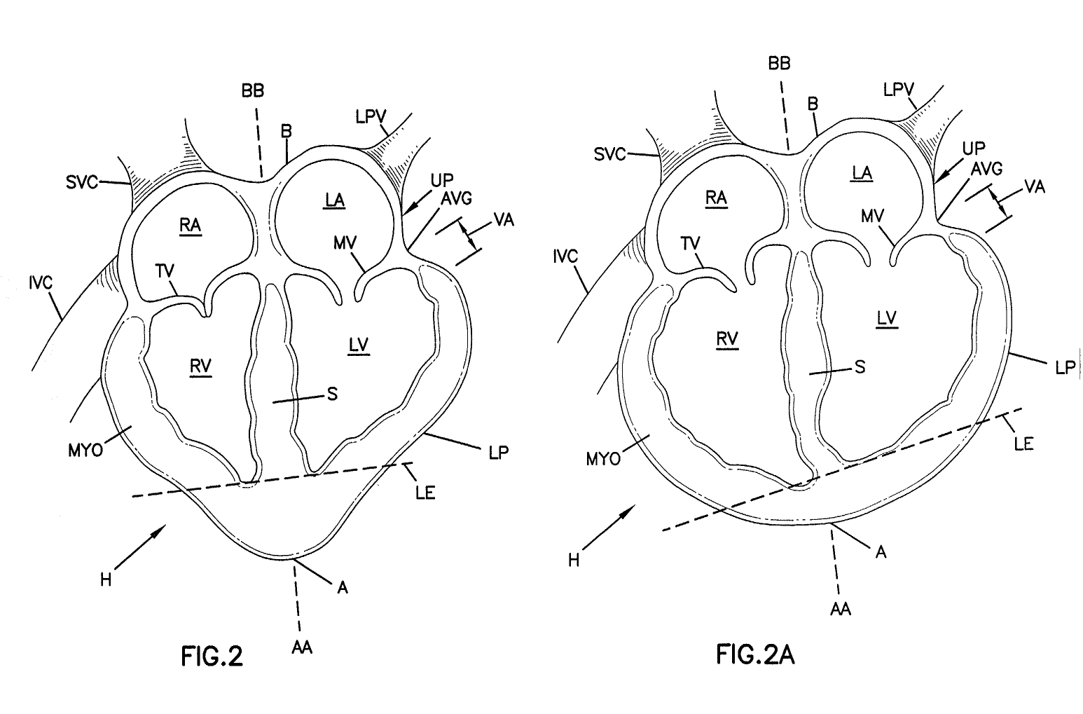

Having described a healthy heart H′ during, systole (FIG. 1) and diastole (FIG. 1A), comparison can now be made with a heart H deformed by congestive heart disease. Such a heart H is shown in systole in FIG. 2 and in diastole in FIG. 2A. All elements of diseased heart H are labeled identically with similar elements of healthy heart H′ except for the omission of the apostrophe in order to distinguish diseased heart H from healthy heart H′.

Comparing FIGS. 1 and 2 (showing hearts H′ and H during systole), the lower portion LP of the diseased heart H has lost the tapered conical shape of the lower portion LP′ of the healthy heart H′. Instead, the lower portion LP of the diseased heart H dilates outwardly between the apex A and the A-V groove AVG. So deformed, the diseased heart H during systole (FIG. 2) resembles the healthy heart H′ during diastole (FIG. 1A). During diastole (FIG. 2A), the deformation of heart H is even more extreme.

As diseased heart H enlarges from the representation of FIGS. 1 and 1A to that of FIGS. 2 and 2A, the heart H becomes a progressively more inefficient pump. Therefore, the heart H requires more energy to pump the same amount of blood. Continued progression of the disease results in the heart H being unable to supply adequate blood to the patient's body, which causes the patient to become symptomatic of cardiac insufficiency.

For ease of illustration, the progression of congestive heart disease has been illustrated and described with reference to a progressive dilation of the lower portion LP of the heart H. While such enlargement of the lower portion LP is most common and troublesome, enlargement of the upper portion UP may also occur.

In addition to cardiac insufficiency, the enlargement of the heart H can also lead to valvular disorders. As the circumference of the valvular annulus VA increases, the leaflets of the tricuspid and mitral valves, TV and MV, may spread apart. After a certain amount of enlargement, the spreading may be so severe that the leaflets cannot completely close. Incomplete closure results in valvular regurgitation contributing to an additional degradation in cardiac performance. While circumferential enlargement of the valvular annulus VA may contribute to valvular dysfunction as described, the separation of the valve leaflets is most commonly attributed to deformation of the geometry of the heart H.

B. Cardiac Support Therapy

Having described the characteristics and problems of congestive heart disease, a treatment method and apparatus are described, such as that disclosed in U.S. Pat. No. 6,085,754, issued Jul. 11, 2000. In general, a jacket is configured to surround the myocardium MYO. While the method of the present invention will be described with reference to a jacket as described in U.S. Pat. No. 6,085,754, it will be appreciated the present invention is applicable to any cardiac support device including those shown in U.S. Pat. No. 5,800,528 and PCT International Publication No. WO 98/29401.

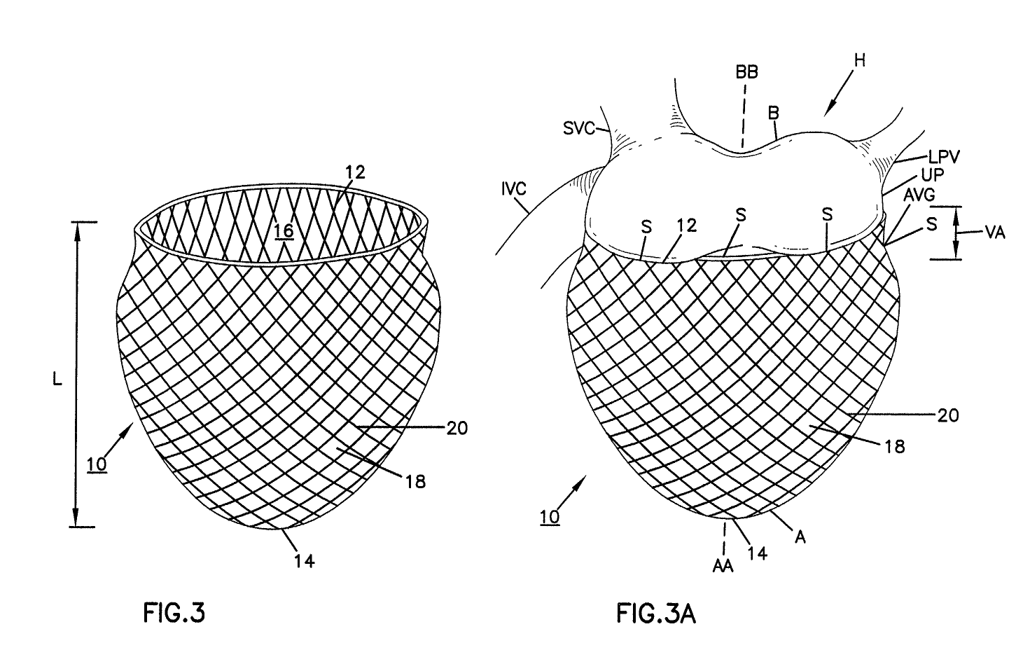

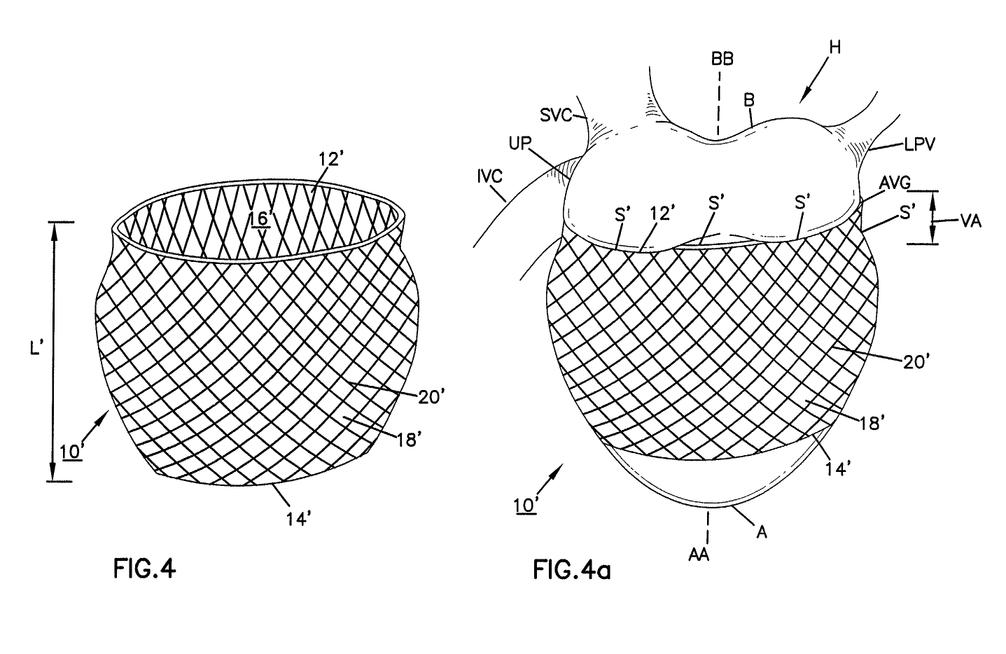

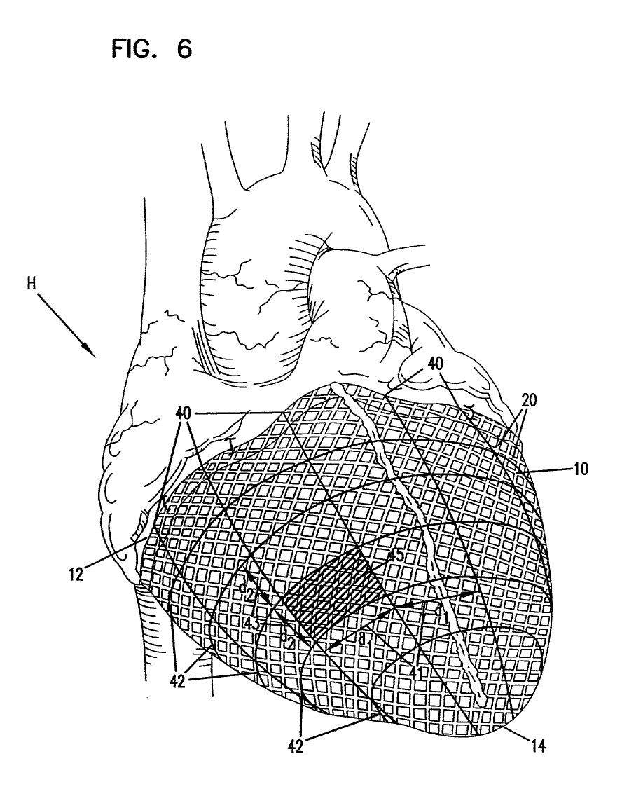

With reference now to FIGS. 3, 3A, 4 and 4A, an example of a cardiac support device is shown as a jacket 10, 10′ of flexible, biologically compatible material. For ease of illustration, visually perceptible tension indictors of the present invention are not shown in FIGS. 3, 3A, 4 and 4A and will be separately described with reference to FIGS. 6, 7, 8, 9, 10, and 11.

The jacket 10, 10′ is an enclosed material having upper and lower ends 12, 12′, 14 and 14′. The jacket 10, 10′ defines an internal volume 16, 16′ which is completely enclosed but for the open ends 12, 12′ and 14′. In the embodiment of FIG. 3, lower end 14 is closed. In the embodiment of FIG. 4, lower end 14′ is open. In both embodiments, upper ends 12 and 12′ are open. Throughout this description, the embodiment of FIG. 3 will be discussed. Elements in common between the embodiments of FIGS. 3 and 4 are numbered identically with the addition of an apostrophe to distinguish the second embodiment and such elements need not be separately discussed.

The jacket 10 is dimensioned with respect to a heart H to be treated. Specifically, the jacket 10 is sized for the heart H to be constrained within the volume 16. The jacket 10 can be slipped around the heart H. The jacket 10 has a length L between the upper and lower ends, 12 and 14, sufficient for the jacket 10 to constrain the lower portion LP. The upper end 12 of the jacket 10 extends at least to the A-V groove AVG and further extends to the lower portion LP to constrain at least the lower ventricular extremities LE.

When the parietal pericardium is opened, the lower portion LP is free of obstructions for applying the jacket 10 over the apex A. If, however, the parietal pericardium is intact, the diaphragmatic attachment to the parietal pericardium inhibits application of the jacket over the apex A of the heart H. In this situation, the jacket 10′ can be opened along a line extending from the upper end 12′ to the lower end 14′ of jacket 10′. The jacket 10′ can then be applied around the pericardial surface of the heart H and the opposing edges of the opened line secured together after placed on the heart H. Systems for securing the opposing edges are disclosed in, for example, U.S. Pat. No. 5,702,343, the entire disclosure of which is incorporated herein by reference. The lower end 14′ can then be attached to the diaphragm or associated tissues using, for example, sutures, staples, or the like.

In the embodiment of FIGS. 3 and 3A, the lower end 14 is closed and the length L is sized for the apex A of the heart H to be received within the lower end 14 when the upper end 12 is placed at the A-V groove AVG. In the embodiment of FIGS. 4 and 4A, the lower end 14′ is open and the length L′ is sized for the apex A of the heart H to protrude beyond the lower end 14′ when the upper end 12′ is placed at the A-V groove AVG. The length L′ is sized so that the lower end 14′ extends beyond the lower ventricular extremities LE such that in both jackets 10, 10′, the myocardium MYO surrounding the ventricles RV, LV is in direct opposition to material of the jacket 10, 10′ during diastole. Such placement is desirable so that the jacket 10, 10′ offers support of the ventricular portions of the heart H. The invention also anticipates and encompasses the placement of more than one open lower end 14′ jackets 10 being placed on a heart H.

After the jacket 10 is positioned on the heart H as described above, the jacket 10 is secured thereto. Preferably, the jacket 10 is secured to the heart H using sutures (or other fastening devices including, but not limited to staples). The jacket 10 is sutured to the heart H at suture locations S circumferentially spaced along the upper end 12. While a surgeon may elect to add additional suture locations to prevent shifting of the jacket 10 after placement, the number of such locations S is preferably limited so that the jacket 10 does not restrict contraction of the heart H during systole.

In one embodiment, the jacket 10 is constructed from a knit material. Preferably the jacket 10 is constructed from a compliant, biocompatible material. As used herein, the term “compliant” refers to a material that can expand in response to a force. “Compliance” refers to the displacement per a unit load for a material. “Elasticity” refers to the ability of the deformed material to return to its initial state after the deforming load is removed. In one embodiment, the jacket 10 material comprises intertwined fibers, for example, fibers intertwined as a knit or weave. In a preferred embodiment, the jacket 10 material is a knit material that may have a degree of compliance and elasticity.

In a preferred embodiment, the fibers are 70 Denier polyester. While polyester is presently preferred, other suitable materials include polytetrafluoroethylene (PTFE), expanded PTFE (ePTFE) and polypropylene.

The knit material has numerous advantages. Such a material is compliant to permit unrestricted movement of the heart H (other than the desired support for the heart H). The material is open defining a plurality of interstitial spaces for fluid permeability as well as minimizing the amount of surface area of direct contact between the heart H and the material of the jacket 10 (thereby minimizing areas of irritation or abrasion) to minimize fibrosis and scar tissue.

The open areas of the knit construction also allow for electrical connection between the heart H and surrounding tissue for passage of electrical current to and from the heart H. For example, although the knit material is an electrical insulator, the open knit construction is sufficiently electrically permeable to permit the use of trans-chest defibrillation of the heart H. Also, the open, compliant construction permits passage of electrical elements (e.g., pacer leads) through the jacket. Additionally, the open construction permits visibility of the epicardial surface, thereby minimizing limitations to performing other procedures, e.g., coronary bypass, to be performed without removal of the jacket 10.

The fabric 18 is preferably tear and run resistant. In the event of a material defect or inadvertent tear, such a defect or tear is restricted from propagation by reason of the knit construction.

The jacket 10 constrains further undesirable circumferential enlargement of the heart H while not impeding other motion of the heart H. With the benefits of the present teachings, numerous modifications are possible. For example, the jacket 10 need not be directly applied to the epicardium (i.e., outer surface of the myocardium) but could be placed over the pericardium. Further, an anti-fibrosis lining (such as a PTFE coating on the fibers of the knit) could be placed between the heart H and the jacket 10. Alternatively, the fibers 20 can be coated with PTFE.

The jacket 10 can be used in the early stages of congestive heart disease. For patients facing heart enlargement due to viral infection, the jacket 10 permits support of the heart H for a sufficient time to permit the viral infection to pass. In addition to preventing further heart enlargement, the jacket 10 treats valvular disorders by constraining circumferential enlargement of the valvular annulus and deformation of the ventricular walls.

C. Tensioning of the Jacket

To permit the jacket 10 to be easily placed on the heart H, the volume and shape of the jacket 10 are typically larger than the lower portion LP during diastole. So sized, the jacket 10 may be easily slipped around the heart H. Once placed around the heart H, the volume and shape of the jacket 10 are adjusted. The jacket 10 is tensioned or adjusted to provide support of heart H.

Support of heart H includes varying levels of tensioning, meant to provide varying levels of support. For example, the jacket 10 could be tensioned to a level that resists further enlargement of the heart, to a level that provides acute wall support, and/or to a level that reduces the size of the heart. As used herein, resisting further enlargement of the heart means resisting expansion or dilation of the heart that would increase the volume of the heart. As used herein, providing acute wall support means reducing stress on the wall of the heart, supporting the internal pressure of the heart, or reducing the transmural wall pressure by offloading the heart. As used herein, reducing the size of the heart means to reduce the volume or left ventricular end-diastolic diameter (LVEDD) of the heart. In general, the level of tension can also be described as applying a load to heart H, or offloading from heart H.

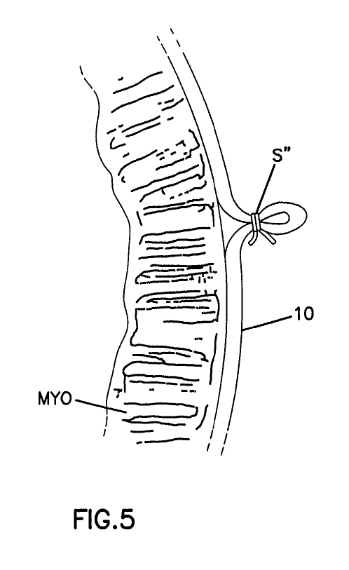

Such variable tensioning is easily accomplished. For example, excess material of the jacket 10 can be gathered and sutured S″ (FIG. 5) to reduce the volume 16 of the jacket 10 and conform the jacket 10 to the shape of the heart H during diastole. Alternatively, the jacket 10 can be gathered as in FIG. 5, and any excess material of the jacket 10 can be removed by cutting. Such shape represents a maximum adjusted volume. This tensioning would allow the jacket 10 to resist enlargement of the heart H beyond the maximum adjusted volume while avoiding restricted contraction of the heart H during systole. As an alternative to the gathering of FIG. 5, the jacket 10 can be provided with other arrangements for adjusting volume. For example, as disclosed in U.S. Pat. No. 5,702,343, the jacket can be provided with a slot. The edges of the slot can be drawn together to reduce the volume 16 of the jacket 10.

If the jacket 10 is to be used to resist further enlargement of heart H, the jacket 10 is generally adjusted for a snug fit of the heart H during diastole without significantly affecting contraction during systole. A snug fit of jacket 10 is defined as the state of jacket 10 when all of the wrinkles have been removed from the jacket 10, but there is little offloading of the heart H. A snug fit can also be defined by a pressure of less than about 10 mm Hg (1.3 kPa) begin exerted on the heart H at diastole by jacket 10. Preferably a snug fit entails a pressure of less than or equal to about 5 mm Hg (0.66 kPa) being exerted on the heart H at diastole. More preferably, a snug fit entails a pressure of less than or equal to about 2 mm Hg (0.27 kPa) being exerted on the heart H at diastole by jacket 10. When the jacket 10 is meant to prohibit further enlargement of the heart H, the jacket 10 will preferably not be significantly offloading the heart H, or will be offloading the heart H only a small amount, until the heart H starts to expand. Preferably, the jacket 10 exerts no or only a slight pressure on the heart H at end systole.

If the jacket 10 is to be used to provide acute wall support, the jacket 10 is generally adjusted to provide more tension than a snug fit, as that term is defined above. In this case, the jacket 10 is first adjusted for a snug fit, then more tension is applied to the jacket 10 to offload the heart H. Preferably, the jacket 10 exerts pressure on the heart H at end diastole. Preferably this pressure at end diastole is between about 1 mm Hg (0.14 kPa) and about 20 mm Hg (2.6 kPa), more preferably between about 5 mm Hg (0.66 kPa) and about 15 mm Hg (2.0 kPa), and most preferably between about 2 mm Hg (0.27 kPa) and about 10 mm Hg (1.3 kPa).

If the jacket 10 is to be used to reduce the size of the heart H, the jacket 10 is generally adjusted to provide more tension than a snug fit, as defined above. In this case, the jacket 10 is first adjusted for a snug fit, then more tension is applied to the jacket 10 in order to reduce the size of the heart H. The amount of tension applied to reduce the size of the heart H may or may not be equal to an amount of tension applied to the heart H to provide acute wall support. Reducing the size of the heart H is similar to providing acute wall support, except that the amount of tension is determined by monitoring dimensional changes in the heart H, instead of the pressure exerted on the heart H. In order to monitor the dimensional changes in the heart H, the volume or the left ventricular end-diastolic diameter (LVEDD) is measured. Preferably, the LVEDD is monitored. The desired decrease in LVEDD will depend on the desired amount of size reduction. Preferably, however, the jacket 10 is adjusted to provide no more than a 10% reduction in LVEDD of heart H.

Care is taken to avoid over tightening the jacket 10 such that cardiac function is impaired. During diastole, the left ventricle LV fills with blood. If the jacket 10 is too tight, the left ventricle LV cannot adequately expand and left ventricular pressure will rise. Overtightening of jacket 10 can be avoided during fitting by monitoring the left ventricular pressure. One example of a well-known technique for monitoring the left ventricular pressure, also called the pulmonary wedge pressure uses a catheter placed in the pulmonary artery. The wedge pressure provides an indication of filling pressure in the left atrium LA and left ventricle LV. While minor increases in pressure (e.g., 2-3 mm Hg) can be tolerated, the jacket 10 is preferably fit on the heart H, so a significant increase in left ventricular pressure during diastole is not observed.

The pressure exerted by the jacket 10 is also important because the wall of the right ventricle RV tends to be thinner than the wall of the left ventricle LV. This tends to cause the pressure in the right ventricle RV to be lower than the pressure in the left ventricle LV. Therefore, the pressure exerted by the jacket 10 on the heart H is preferably equal or less than the end diastolic pressure of the right ventricle RV. If the pressure exerted by the jacket 10 is greater than the end diastolic pressure of the right ventricle RV, expansion and/or filling of the right ventricle RV may be compromised. Generally, a fitted jacket 10 that causes less than a 10% reduction in maximum diastolic dimension (e.g. LVEDD) serves to reduce cardiac volume without compromising cardiac function. If the jacket 10 does cause excessive pressure on the heart H, decreases in cardiac output, increased central venous pressure, and/or decreased systolic pressure may all be seen.

To facilitate a surgeon's ease of obtaining a desired amount of pressure on the heart H, the jacket 10 includes indicators to monitor the tensioning of the jacket 10. The indicators of the invention can allow for monitoring of the tension applied to the jacket 10 and/or for tensioning of the jacket 10 to at least one predetermined level of constraint.

1. Indicator Grid Pattern

One embodiment of the invention, illustrated in FIG. 6, comprises jacket 10, length markers 40, width markers 42, width gap 41, length gap 43, and grid area 45.

Length markers 40 have an orientation generally parallel along the longitudinal axis (from upper end 12, to lower end 14, of jacket 10). Width markers 42 have a general orientation perpendicular to length markers 40. Length markers 40 and width markers 42 generally form a grid on or in jacket 10.

Length markers 40 and width markers 42 define width gap 41 and length gap 43 respectively. Width gap 41 is defined by the distance (d₁) between any two adjacent length markers 40. Length gap 43 is defined by the distance (d₂) between any two adjacent width markers 42. Width gap 41 and length gap 43 may be, but need not be equal. Grid area 45 is defined as the area enclosed by two adjacent length markers 40 and two adjacent width markers 42. A numerical value of grid area 45 can be determined by multiplying the dimensions of width gap 41 and length gap 43.

Length markers 40 and width markers 42 can be constructed of any material that allows them to be visually perceived on or in the jacket 10. For example, length markers 40 and width markers 42 can be visually perceived if a different color than jacket 10, a different fiber than jacket 10, or a different texture than jacket 10. The material of length markers 40 and width markers 42 should also be chosen so as to not have a substantial detrimental effect on the heart H or the functioning of jacket 10. Examples of substantial detrimental effects include but are not limited to adverse tissue reactions, disruptions in the uniform distribution of stress on the jacket 10, and effects on compliance. What does compliance really mean. The material of length markers 40 and width markers 42 can either be expandable or non-expandable.

Examples of materials that can be used for length markers 40 and width markers 42 include but are not limited to sutures, dyes or fibers. Examples of materials that could be used as sutures include but are not limited to polyester (PET), polytetrafluoroethylene (PTFE) or other biocompatible materials. Examples of dyes that could be applied to jacket 10 as length markers 40 and width markers 42 include but are not limited to biocompatible dyes or pigments that may be embedded in the jacket fibers. Examples of fibers that can be used for length markers 40 and width markers 42 include but are not limited to polyester, polytetrafluoroethylene (PTFE), expanded PTFE (ePTFE), polypropylene or other biocompatible polymers.

If length markers 40 and width markers 42 comprise fibers, they can be constructed in a number of different ways. An example of one method includes weaving them into the material of jacket 10 and securing them to jacket 10. Length markers 40 and width markers 42 can be secured to jacket 10 by for example, suturing length markers 40 and width markers 42 to the fibers 20 of jacket 10. Alternatively, length markers 40 and width markers 42 could be constructed of the fibers 20 of the jacket 10 itself by selectively dying areas of the fibers 20.

In this embodiment, grid area 45 indicates the tensioning of the jacket 10. The embodiment of the invention illustrated in FIG. 6 is in a relaxed state. As used herein, the phrase relaxed state, or the phrase not tensioned refers to the jacket 10 when placed upon the heart and snugly fit on heart H, as snug fit is defined above. The jacket 10 in a relaxed state will have a value of grid area 45 that is equal to the dimension of length gap 41 (d₁) multiplied by the dimension of width gap 43 (d₂).

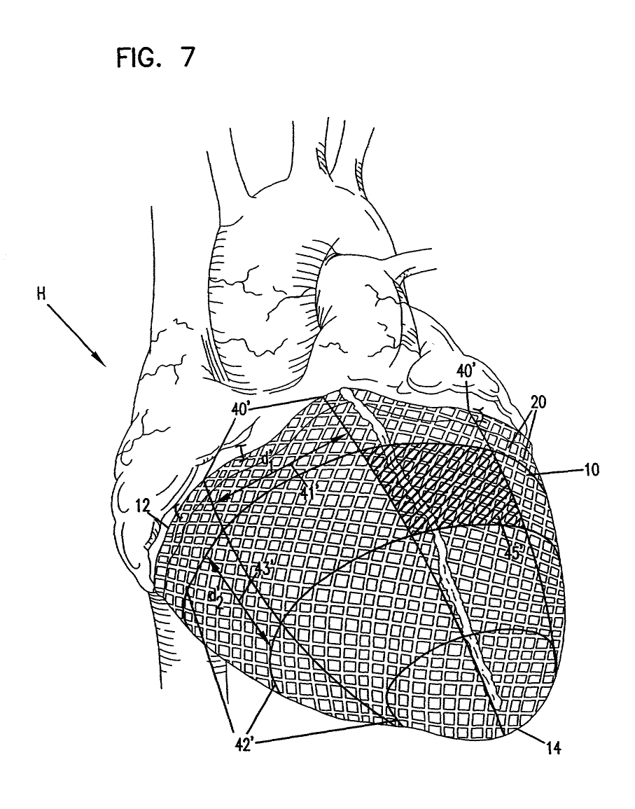

Referring to FIG. 7, as jacket 10 is tensioned to support heart H the material of jacket 10 is expanded. As jacket 10 is tensioned, it goes from having a snug fit to applying a load to the heart H to offload the wall of the heart H. The more jacket 10 is tensioned, the more load is applied to the heart H. The tension indicators of the present invention indicate this load through increases in the dimension of the width gap 41′ (d₁′) and the dimension of the length gap 43′ (d₂′), and the grid area 45′. The more load applied or conversely, the more load offloaded from the heart H, the greater the dimension of the width gap 41′ (d₁′), the dimension of the length gap 43′ (d₂′), grid area 45′ and combinations thereof become. In general, the higher the load on the jacket 10, the lower the stress on the wall of heart H for a given internal pressure.

The level of support of heart H can be monitored by for example monitoring grid area 45′. As grid area 45′ increases, the level of support of the heart H also increases. The value of grid area 45′ can either be used as a rough estimate of the level of support of the heart H, or specific values of grid area 45′ can be calibrated to correspond with the level of support of the heart H provided thereby. For example a value of grid area 45′ could correlate with a given percentage of constraint of cardiac expansion of the heart H.

The specific values of grid area 45′ that would correlate with specific levels of support of the heart H would depend on the spacing of the individual fibers 20 of jacket 10 and the spacing and extendibility of length markers 40 and width markers 42. Such values could be easily determined by one of skill in the art. A jacket 10 could have numerous predetermined values of grid area 45′ that correlated with specific levels of support of the heart H.

Length markers 40 and width markers 42 can be but need not be configured so as to be detachable from jacket 10. Once jacket 10 has been tensioned to provide the desired level of support of the heart H, the length markers 40 and width markers 42 can remain in or on jacket 10 or alternatively can be detached therefrom.

2. Indicator Fit Lines

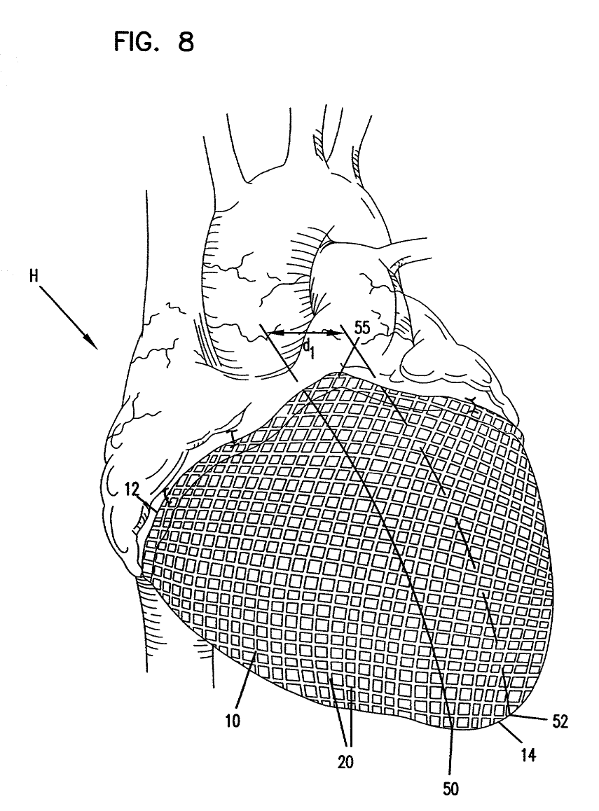

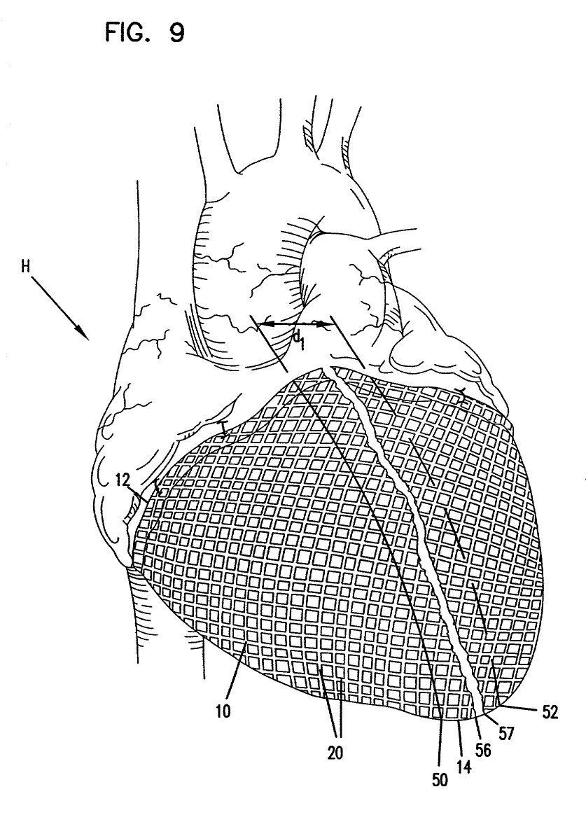

Another embodiment of the invention, illustrated in FIGS. 8 through 12 comprises jacket 10, baseline marker 50 and at least a first fit line marker 52. In one embodiment, the jacket 10 is closed and may comprise excess material 55 as seen in FIG. 8. In another embodiment, jacket 10 is open and may comprise first fabric end 56 and second fabric end 57, as seen in FIG. 9.

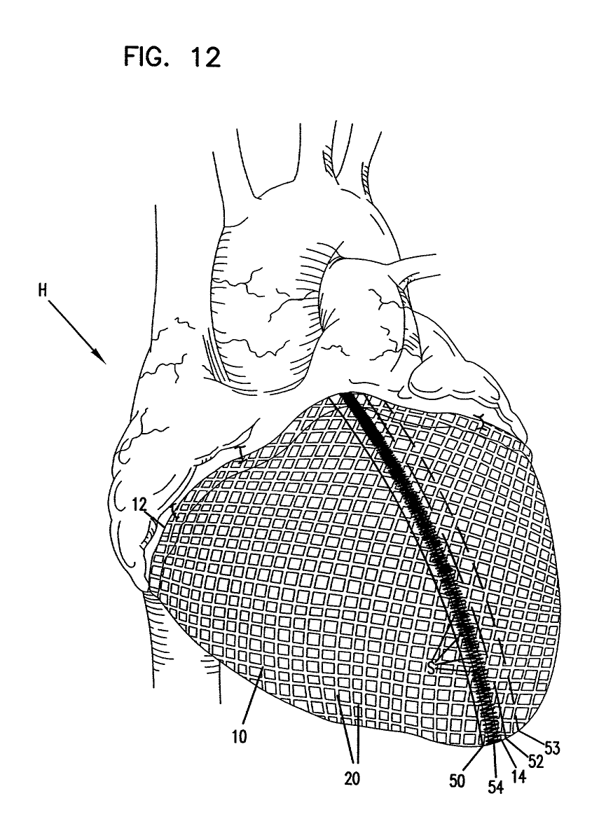

Baseline marker 50 and first fit line marker 52 are generally oriented parallel to the longitudinal axis (i.e. extending from upper end 12, to lower end 14,) of jacket 10. Baseline marker 50 and first fit line marker 52 may, but need not, extend the entire length of the longitudinal axis of jacket 10. Preferably, baseline marker 50 and first fit line marker 52 extend along most, if not all, the longitudinal axis of jacket 10. In one embodiment there are at least two indicator lines, first fit line marker 52 and second fit line marker 53, as seen in FIG. 12.

Baseline marker 50 and first fit line marker 52 can be constructed of any material that allows them to be visually perceived on or in the jacket 10. For example baseline marker 50 and first fit line marker 52 can be visually perceived if a different color than jacket 10, a different fiber than jacket 10, or a different texture than jacket 10. The material of baseline marker 50 and first fit line marker 52 should also be chosen so as to not have a substantial detrimental effect on the heart H or the functioning of jacket 10. Examples of substantial detrimental effects include but are not limited to adverse tissue reactions, disruptions in the uniform distribution of stress on the jacket 10, and effects on compliance.

Examples of materials that can be used for baseline marker 50 and first fit line marker 52 include but are not limited to sutures, dyes, fibers. Examples of materials that could be used as sutures include but are not limited to polyester (PET), polytetrafluoroethylene (PTFE) or other biocompatible materials. Examples of dyes that could be applied to jacket 10 as fit line markers, such as first fit line marker 52 include but are not limited to biocompatible dyes or pigments that may be embedded in the jacket fibers. Examples of fibers that can be used for baseline marker 50 and first fit line marker 52 include but are not limited to polyester, polytetrafluoroethylene (PTFE), expanded PTFE (ePTFE), polypropylene or other biocompatible polymers.

If baseline marker 50 and first fit line marker 52 comprise fibers, they can be constructed in a number of different ways. An example of one method includes weaving them into the material of jacket 10 and securing them to jacket 10. Baseline marker 50 and first fit line marker 52 can be secured to jacket 10 by for example, suturing them to the fibers 20 of jacket 10. Alternatively, baseline marker 50 and first fit line marker 52 could be constructed of the fibers 20 of the jacket 10 itself by selectively dying areas of the fibers 20.

When jacket 10 is first placed on the heart H, jacket 10 may have excess material 55. Excess material 55 as used herein (and illustrated in FIG. 8) refers to material of jacket 10 that can be gathered and pulled away from the heart H as jacket 10 is fit to conform to the surface of heart H giving a snug fit, as that phrase was defined above. If excess material 55 is present, the jacket 10 is generally gathered so that the excess material 55 is positioned between baseline 50 and fit line 52 (see FIG. 8). The distance between baseline 50 and first fit line marker 52, d₁ is the baseline distance in a relaxed condition.

FIG. 9 illustrates another embodiment of the invention when first placed upon heart H. In this embodiment the jacket 10 is open along a line extending from the upper end 12 to the lower end 14 of jacket 10. This creates first fabric end 56 and second fabric end 57. Opening jacket 10 in this manner allows it to be applied around the pericardial surface of the heart H and then subsequently closed by securing first fabric end 56 and second fabric end 57 together.

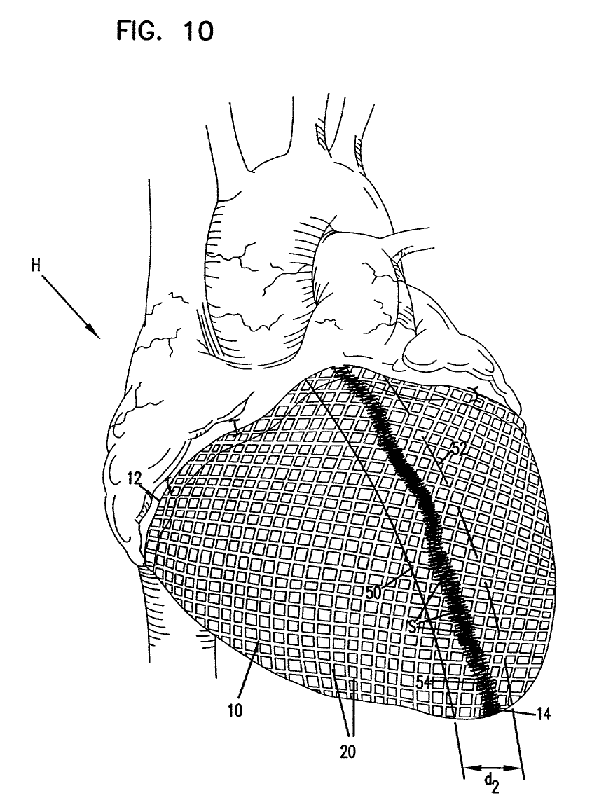

In FIG. 10, a jacket 10, as depicted either in FIGS. 8 or 9, is tensioned to a snug fit, and a closure 54 is made between the baseline 50 and fit line 52. Closure 54 can be formed by making sutures S, as illustrated in FIG. 5. Excess material 55 can then be removed. Once the excess material 55 has been removed, the loose ends of jacket 10 may optionally be incorporated into closure 54. The fit of jacket 10 after the above process can be indicated in part by a reduced distance from baseline 50 to first fit line marker 52, d₂ as compared with the baseline distance d, (shown in FIGS. 8 and 9).

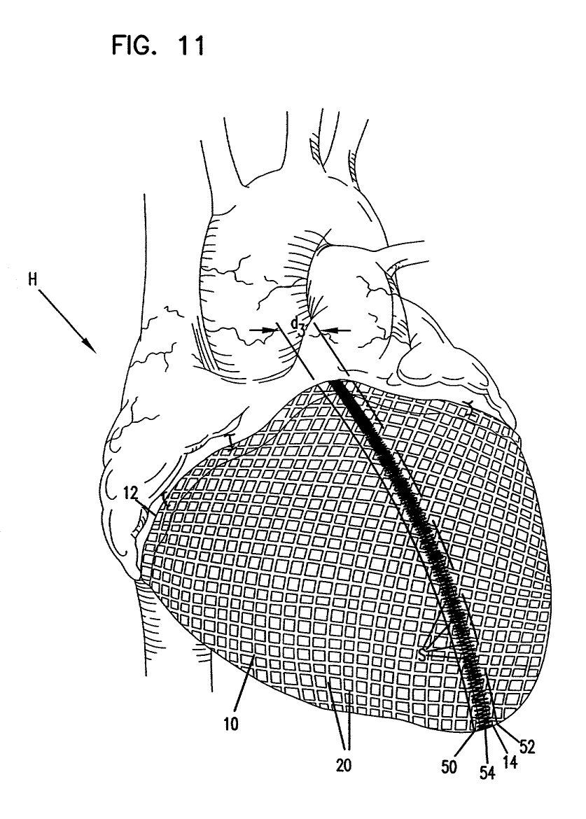

FIG. 11 illustrates a jacket 10, as depicted in FIGS. 8 or 9, tensioned tighter than the jacket 10 in FIG. 10. A higher level of tension in the jacket of FIG. 11 (as compared to the jacket of FIG. 9) is indicated by the distance from baseline 50 to fit line marker 52, d₃ in FIG. 11. The level of tension in FIG. 10 is indicated by the distance from baseline 50 to first fit line marker 52, d₂. The jacket in FIG. 11 has a higher level of tension than that in FIG. 10 because d₃ is less than d₂.

The tensioning of jacket 10 to a desired level can be accomplished in one tensioning/closure 54 forming step, or multiple steps. For example, jacket 10 can be placed on heart H, the excess material 55, if present, gathered, sutures put in place to maintain the given level of tension, and then the jacket can be tensioned further and closure 54 made. In another example, jacket 10 can be placed on heart H, excess material 55, if present, gathered, sutures put in place to maintain the given level of tension, a wedge pressure measurement of LVEDD measurement taken, the jacket 10 tensioned further, sutures put in place to maintain the given level of tension, a wedge pressure or LVEDD measurement taken, then closure 54 made. It should be understood that any combination or order of these and other steps known to those of skill in the art can be utilized to obtain the desired tensioning of jacket 10.

The material of the jacket 10 between baseline marker 50 and first fit line marker 52 need not be but can be gathered similarly along the length of the jacket 10. Irregular shapes of heart H may make irregular gathering of this material desirous. The present invention may be utilized in this, or other similar ways. Alternatively, if a certain region of a heart H requires more support than other regions, certain parts of jacket 10 may be tensioned more.

Jacket 10 may alternatively be configured with more than one fit line marker. FIG. 12 illustrates an embodiment of the invention that further comprises second fit line marker 53.

The fit line markers, for example first fit line marker 52 and/or second fit line marker 53, may further aid a surgeon in estimating how much the jacket 10 needs to be tensioned. For example, the jacket 10 could be tensioned as dictated by a first fit line marker 52, then the wedge pressure, or the LVEDD could be measured, and the tension adjusted further according to second fit line marker 53. It should also be understood that the jacket 10 can be tensioned using any combination of these steps. The fit line markers provide the surgeon with an estimate of the final tensioning of the jacket 10. Such uses of jacket 10 may result in a tensioned state in which one or more fit lines have either been removed from jacket 10 as excess material 55 or have been incorporated into closure 54. Alternatively, if irregular heart shapes or irregular levels of tensioning are desired across the heart, the fit line markers can provide estimates at some regions along jacket 10 and not others. Irregular heart shapes could also result in one or more fit lines being removed as excess material 55 or incorporated into closure 54 at some regions of the heart H and not at others.

Once jacket 10 has been tensioned to provide the desired level of cardiac support for heart H, the markers (baseline marker 50, first fit line marker 52, second fit line marker 53, etc.) can remain in or on jacket 10 or alternatively can be detached therefrom.

From the foregoing detailed description, the invention has been described in a preferred embodiment. Modifications and equivalents of the disclosed concepts are intended to be included within the scope of the appended claims.

What is claimed is:

1. A device for providing support to a heart having a longitudinal axis from an apex to a base and having an upper portion and a lower portion divided by an A-V groove, the device comprising:
a jacket of flexible material of open cell construction defining a volume between an open upper end and a lower end, said jacket dimensioned for said apex of said heart to be inserted into said volume through said open upper end and for said jacket to be slipped over said heart, said jacket further dimensioned for said jacket to have a longitudinal dimension between said upper and lower ends sufficient for said jacket to support said lower portion;
said jacket adapted to be adjusted on said heart to snugly conform to an external geometry of said heart and assume a maximum adjusted volume for said jacket to support said heart;
an indicator for indicating when said jacket is adjusted on said heart to a desired level of support of said heart, said indicator comprising at least one marker positioned substantially parallel to the longitudinal dimension of said jacket.

2. The device of claim 1 wherein said support of said heart applies a load to said heart.

3. The device of claim 1, where said support of said heart provides offloading of said heart.

4. The device of claim 1, wherein said support of said heart provides a desired level of cardiac constraint to said heart.

5. The device of claim 1, wherein said indicator is detachable from said jacket material.

6. The device of, claim 1, wherein said indicator is constructed from said jacket material.

7. The device of claim 1, wherein said jacket has a knit construction.

8. The device of claim 1, wherein said markers have a perceptibly different color than said jacket material.

9. The device of claim 1, wherein said indicator further comprises markers perpendicular to the longitudinal axis.

10. The device of claim 9 wherein said markers along the longitudinal axis and said markers perpendicular thereto form a grid pattern.

11. The device of claim 10, wherein said markers define a grid area.

12. The device of claim 11, wherein said device in a relaxed state has a smaller grid area than said grid area when said device is tensioned to provide support to a heart.

13. The device of claim 9, wherein said markers comprise polyester, polytetrafluoroethylene, expanded polytetrafluoroethylene, polypropylene, or combinations thereof.

14. The device of claim 9, wherein said markers are constructed from polyester.

15. The device of claim 14, wherein said polyester further comprises a biocompatible dye.

16. The device of claim 1, wherein said indicator further comprises at least one other marker positioned substantially parallel to the longitudinal dimension of said jacket.

17. The device of claim 16, wherein one marker functions to indicate a relaxed state and at least one marker functions to indicate a level of support on said heart.

18. The device of claim 16, wherein said markers comprise polyester, polytetrafluoroethylene, expanded polytetrafluoroethylene, polypropylene, or combinations thereof.

19. The device of claim 18, wherein said markers are constructed from polyester.

20. The device of claim 19, wherein said polyester further comprises a biocompatible dye.
